(19) United States
(12) Patent Application Publication
(10) Pub. No.: US 2020/0345245 A1
(43) Pub. Date: Nov. 5, 2020

(54) MEASUREMENT APPARATUS AND COMPUTER-READABLE RECORDING MEDIUM

(71) Applicants: OMRON HEALTHCARE CO., LTD., Muko-shi (JP); OMRON CORPORATION, Kyoto-shi (JP)

(72) Inventors: Kentaro MORI, Kyoto (JP); Yasuhiro KAWABATA, Kyoto (JP); Naomi MATSUMURA, Kyoto (JP)

(73) Assignees: OMRON HEALTHCARE CO., LTD., Muko-shi (JP); OMRON CORPORATION, Kyoto-shi (JP)

(21) Appl. No.: 16/899,450

(22) Filed: Jun. 11, 2020

Related U.S. Application Data
(63) Continuation of application No. PCT/JP2018/044217, filed on Nov. 30, 2018.

(30) Foreign Application Priority Data
Dec. 21, 2017 (JP) 2017-245311

(51) Int. Cl. A61B 5/021; A61B 5/00; A61B 5/0235

(52) U.S. Cl. CPC A61B 5/02108; A61B 5/681; A61B 5/7225; A61B 5/0235; A61B 5/0022; A61B 5/02141; A61B 5/742

(57) ABSTRACT

An apparatus includes a first pulse wave sensor and a second pulse wave sensor that can be arranged in correspondence with respective measurement sites distant from each other. The first pulse wave sensor outputs a current signal having a first frequency to a measurement site and detects a voltage signal that represents pulse waves from the measurement site, based on a filter characteristic corresponding to the first frequency. The second pulse wave sensor outputs a current signal having a frequency different from the first frequency to a corresponding measurement site and detects a voltage signal representing pulse waves from the measurement site, based on a filter characteristic corresponding to a second frequency.

### CROSS REFERENCE TO RELATED APPLICATIONS

The present application is a continuation of International application No. PCT/JP2018/044217, filed Nov. 30, 2018, which claims priority to Japanese Patent Application No. 2017-245311, filed Dec. 21, 2017, the entire contents of each of which are incorporated herein by reference.

### BACKGROUND OF THE INVENTION

### Field of the Invention

The present disclosure relates to a measurement apparatus and a computer-readable recording medium and particularly to an apparatus and a computer-readable recording medium for measuring information on pulse waves.

### Description of the Background Art

For example, Japanese Patent Laying-Open No. 2017-070739 discloses as a method of detecting pulse waves, a configuration that measures a biological signal including information on pulse waves from one or both of a radial artery and an ulnar artery.

Japanese Patent Laying-Open No. 2016-135261 discloses a configuration in which, for detection of pulse waves, light is emitted to a surface of a living body from a sensor including light emitting elements aligned in a first direction and light that has passed through the living body is received at a light reception element and detected as a pulse wave signal. Japanese Patent Laying-Open No. 2016-135261 discloses a configuration that distinguishes between signals of light derived from sensors arranged in proximity by shifting a cycle of light emission from the sensors.

Conventionally, in order to detect information on pulse waves, a configuration that detects pulse wave signals with pulse wave sensors at two different points above an artery has been proposed. When the pulse wave sensors are arranged in proximity in this case, a detection signal from one pulse wave sensor may interfere with a detection signal from the other pulse wave sensor. Therefore, for accurate detection of information on pulse waves, elimination of influence by interference has been desired.

### SUMMARY OF THE INVENTION

An object in one aspect of the present disclosure is to provide a measurement apparatus and a computer-readable recording medium for more accurately obtaining information on pulse waves.

According to one aspect of this disclosure, an apparatus that measures pulse waves includes a first pulse wave sensor unit and a second pulse wave sensor unit that can be arranged in correspondence with respective measurement sites distant from each other.

The first pulse wave sensor unit includes a first output unit that outputs a first current signal having a first frequency to a corresponding measurement site and a first detector that detects a voltage signal representing pulse waves from the corresponding measurement site. The second pulse wave sensor unit includes a second output unit that outputs a second current signal having a second frequency different from the first frequency to a corresponding measurement site and a second detector that detects a voltage signal representing pulse waves from the corresponding measurement site.

The first detector processes the detected voltage signal representing pulse waves based on a filter characteristic corresponding to the first frequency and the second detector processes the detected voltage signal representing pulse waves based on a filter characteristic corresponding to the second frequency.

Preferably, 60 kHz is defined as the first frequency and 50 kHz is defined as the second frequency.

An apparatus that measures pulse waves according to another aspect of this disclosure includes a first pulse wave sensor unit and a second pulse wave sensor unit that can be arranged in correspondence with respective measurement sites distant from each other.

The first pulse wave sensor unit includes a first output unit that outputs a first current signal having a first frequency to a corresponding measurement site and a first detector that detects a voltage signal representing pulse waves from the corresponding measurement site, and the second pulse wave sensor unit includes a second output unit that outputs a second current signal having a second frequency to a corresponding measurement site and a second detector that detects a voltage signal representing pulse waves from the corresponding measurement site. The measurement apparatus alternately drives the first pulse wave sensor unit and the second pulse wave sensor unit at predetermined intervals.

Preferably, the first frequency and the second frequency are equal to each other.

Preferably, the first frequency is different from the second frequency.

Preferably, 50 kHz or 60 kHz is defined as the first frequency and 50 kHz or 60 kHz is defined as the second frequency.

Preferably, the measurement apparatus further detects a pulse wave velocity from at least one of the pulse waves represented by the voltage signal detected by the first detector and the pulse waves represented by the voltage signal detected by the second detector.

Preferably, the measurement apparatus further includes a blood pressure calculator that calculates at least one of a first blood pressure based on the pulse wave velocity calculated based on the pulse waves represented by the voltage signal detected by the first detector and a second blood pressure based on the pulse wave velocity calculated based on the pulse waves represented by the voltage signal detected by the second detector.

Preferably, the measurement apparatus detects an S/N ratio for each of the voltage signals representing the pulse waves and detected by the first detector and the second detector.

Preferably, the blood pressure calculator calculates a blood pressure based on the pulse wave velocity calculated based on the pulse waves represented by a voltage signal higher in S/N ratio, of the voltage signals representing the pulse waves and detected by the first detector and the second detector.

Preferably, the blood pressure calculator calculates a representative blood pressure, of the first blood pressure and the second blood pressure.

Preferably, the representative blood pressure includes an average blood pressure of the first blood pressure and the second blood pressure.

Preferably, the average blood pressure is represented as an average calculated with the first blood pressure and the second blood pressure being weighted, and a weight for the first blood pressure is based on a corresponding S/N ratio and a weight for the second blood pressure is based on a corresponding S/N ratio.

Preferably, the measurement apparatus further includes a display and a communication unit that communicates with an external information processing apparatus including a display unit, and the measurement apparatus transmits a blood pressure value calculated by the blood pressure calculator through the communication unit to the information processing apparatus for display on the display unit.

Yet another aspect of this disclosure is directed to a program that causes a computer to perform a method of controlling an apparatus. The apparatus includes a first pulse wave sensor unit and a second pulse wave sensor unit that can be arranged in correspondence with respective measurement sites distant from each other. The method includes a first output step of controlling the first pulse wave sensor unit to output a first current signal having a first frequency to a corresponding measurement site, a first detection step of controlling the first pulse wave sensor unit to detect a voltage signal representing pulse waves from a measurement site corresponding to the first pulse wave sensor unit, a second output step of controlling the second pulse wave sensor unit to output a second current signal having a second frequency to a corresponding measurement site, a second detection step of controlling the second pulse wave sensor unit to detect a voltage signal representing pulse waves from a measurement site corresponding to the second pulse wave sensor unit, a first processing step of processing the voltage signal representing the pulse waves and detected in the first detection step based on a filter characteristic corresponding to the first frequency, and a second processing step of processing the voltage signal representing the pulse waves and detected in the second detection step based on a filter characteristic corresponding to the second frequency.

According to yet another aspect of this disclosure, a program that causes a computer to perform a method of controlling an apparatus is provided. The apparatus includes a first pulse wave sensor unit and a second pulse wave sensor unit that can be arranged in correspondence with respective measurement sites distant from each other. The method includes a first output step of controlling the first pulse wave sensor unit to output a first current signal having a first frequency to a corresponding measurement site, a first detection step of controlling the first pulse wave sensor unit to detect a voltage signal representing pulse waves from the corresponding measurement site, a second output step of controlling the second pulse wave sensor unit to output a second current signal having a second frequency to a corresponding measurement site, a second detection step of controlling the second pulse wave sensor unit to detect a voltage signal representing pulse waves from the corresponding measurement site, and alternately driving the first pulse wave sensor unit and the second pulse wave sensor unit at predetermined intervals.

The foregoing and other objects, features, aspects and advantages of the present invention will become more apparent from the following detailed description of the present invention when taken in conjunction with the accompanying drawings.

### BRIEF DESCRIPTION OF DRAWINGS

FIG. 1 is a perspective view showing appearance of a blood pressure monitor 1 according to a first embodiment.

FIG. 2 is a diagram schematically showing a cross-section perpendicular to a longitudinal direction of a left wrist 90 with blood pressure monitor 1 according to the first embodiment being attached to wrist 90.

FIG. 3 is a diagram showing a two-dimensional layout of an electrode group for impedance measurement with blood pressure monitor 1 according to the first embodiment being attached to wrist 90.

FIG. 4 is a diagram showing a block configuration of a control system of blood pressure monitor 1 according to the first embodiment.

FIGS. 5A and 5B are diagrams showing a circuit configuration of a sensor unit according to the first embodiment.

FIGS. 6A and 6B are schematic diagrams for illustrating measurement of a blood pressure based on a pulse wave transit time according to the first embodiment.

FIG. 7 is a schematic cross-sectional view along the longitudinal direction of wrist 90 with blood pressure monitor 1 being attached to the wrist in measurement of a blood pressure with an oscillometric method according to the first embodiment.

FIG. 8 is a diagram schematically showing a configuration of a function relating to measurement provided by a CPU 100 according to the first embodiment.

FIG. 9 is a flowchart showing processing in measurement of a blood pressure based on a PTT according to the first embodiment.

FIG. 10 is a diagram showing exemplary storage of a result of measurement according to the first embodiment.

FIG. 11 is a diagram showing exemplary representation of a result of measurement according to the first embodiment.

FIG. 12 is a diagram showing a schematic configuration of a system according to the first embodiment.

FIGS. 13A, 13B and 13C are diagrams for illustrating backgrounds of the first embodiment.

FIG. 14 is a diagram showing a configuration of the first embodiment.

FIG. 15 is a diagram schematically showing a configuration of a function relating to measurement provided by a CPU 100A according to a second embodiment.

FIG. 16 is a diagram schematically showing a cycle CR according to the second embodiment.

FIGS. 17A and 17B are diagrams schematically showing a waveform of a current signal output to a measurement site according to the second embodiment.

FIG. 18 is a flowchart showing a method of controlling blood pressure monitor 1 according to a fourth embodiment.

FIG. 19 is a flowchart showing another method of controlling blood pressure monitor 1 according to the fourth embodiment.

### DESCRIPTION OF EMBODIMENTS

An embodiment of the present invention will be described below with reference to the drawings. The same elements in the description below have the same reference characters allotted and their labels and functions are also identical. Therefore, detailed description thereof will not be repeated.

Though a pulse wave transit time (which will be referred to as a PTT below) is exemplified below as information relating to pulse waves, information on pulse waves is not limited to the PTT. An example in which a measurement apparatus that obtains information on pulse waves is mounted on a blood pressure monitor which is a wearable terminal will be described. An apparatus on which the “measurement apparatus” is mounted is not limited to the blood pressure monitor. The blood pressure monitor is not limited to a wearable terminal.

### First Embodiment

<Configuration of Blood Pressure Monitor>

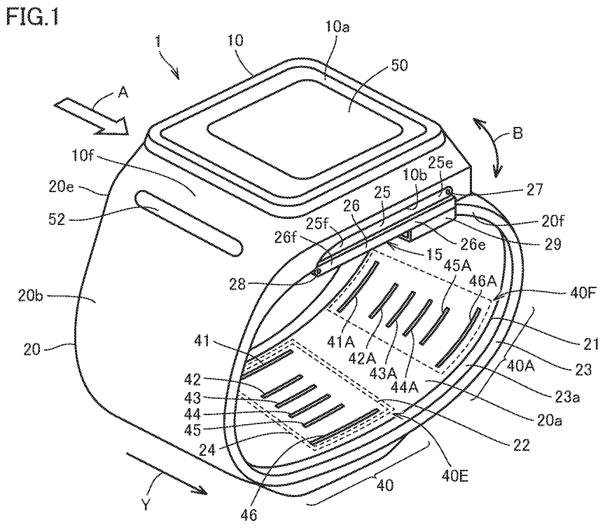

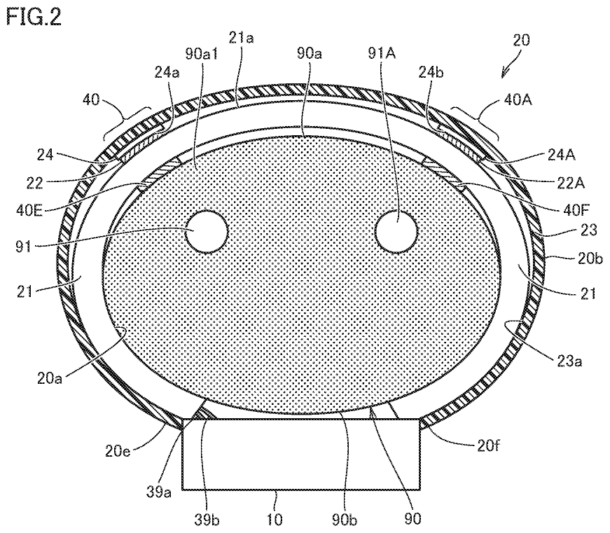

FIG. 1 is a perspective view showing appearance of a blood pressure monitor 1 according to a first embodiment. FIG. 2 is a diagram schematically showing a cross-section perpendicular to a longitudinal direction of a left wrist 90 with blood pressure monitor 1 according to the first embodiment being attached to wrist 90 (which will also be referred to as an “attached state” below). In the present embodiment, left wrist 90 is defined as a measurement site. The “measurement site” should only be a site through which an artery passes, and it is not limited to the wrist. For example, a right wrist, an upper arm, or a lower limb such as an ankle and a thigh may be defined as the measurement site.

Referring to FIGS. 1 and 2, a belt 20 is a band-shaped member. In the attached state, belt 20 is slidably attached by wrapping around, with a longitudinal direction thereof being brought in correspondence with a circumferential direction of wrist 90. Belt 20 has a dimension in a width direction Y (width dimension), for example, of approximately 30 mm. Belt 20 includes a band-shaped body 23 and a compression cuff 21. Band-shaped body 23 includes an inner circumferential surface 23a which is a surface on a side of the measurement site and an outer circumferential surface 20b which is a surface opposite to inner circumferential surface 23a. When belt 20 is attached to the measurement site by wrapping around in the first embodiment, blood pressure monitor 1 is in the “attached state.” “Remaining attached” means that the “attached state” is maintained.

Compression cuff 21 is attached along inner circumferential surface 23a of band-shaped body 23 and includes an inner circumferential surface 20a in contact with wrist 90 (see FIG. 2). Compression cuff 21 is formed as a fluid bag with two stretchable polyurethane sheets being opposed to each other in a thickness direction and with a peripheral portions thereof being molten and joined. In the present embodiment, the fluid bag of compression cuff 21 should only be a member like a bag that can accommodate fluid. Compression cuff 21 is expanded when it is supplied with fluid, and with expansion, the measurement site is pressurized. As the fluid is ejected, compression cuff 21 contracts and a pressurized state of the measurement site is canceled.

A main body 10 is provided integrally with one end 20e of belt 20. Alternatively, belt 20 and main body 10 may separately be formed and main body 10 may be attached integrally with belt 20 with an engagement member (for example, a hinge) being interposed. In the present embodiment, a site where main body 10 is arranged corresponds to a rear-side surface (a surface on a dorsal side) of wrist 90 in the attached state (see FIG. 2). FIG. 2 shows a radial artery 91 and an ulnar artery 91A that pass within wrist 90 in the vicinity of a palm-side surface (a surface on a side of the palm) 90a.

As shown in FIG. 1, main body 10 is in a three-dimensional shape having a thickness in a direction perpendicular to outer circumferential surface 20b of belt 20. Main body 10 is formed to be small in size and thickness so as not to interfere daily activities of a user. Main body 10 has a contour in a shape of a frustum of a pyramid that protrudes outward from belt 20.

A display 50 is provided on a top surface (a surface farthest from a measurement site) 10a of main body 10. An operation portion 52 for inputting an instruction from a user is provided along a side surface (a side surface on a front left side in FIG. 1) 10f of main body 10.

Sensor units 40 and 40A are provided on inner circumferential surface 20a of belt 20 (that is, inner circumferential surface 20a of compression cuff 21) at sites between one end 20e and the other end 20f of belt 20. Sensor units 40 and 40A perform a function to detect pulse waves by using an impedance measurement function.

An electrode group 40E is arranged on inner circumferential surface 20a at the site where sensor unit 40 is arranged. Electrode group 40E includes six plate-shaped (or a sheet-shaped) electrodes 41 to 46 arranged as being distant from one another in width direction Y of belt 20. The site where electrode group 40E is arranged corresponds to radial artery 91 in wrist 90 in the attached state.

A solid material 22 is arranged at a position corresponding to electrode group 40E on an outer circumferential surface 21a. A pressure cuff 24 is arranged on an outer circumferential side of solid material 22. Pressure cuff 24 is an expansion member that locally presses a region corresponding to electrode group 40E in the circumferential direction of compression cuff 21. Pressure cuff 24 is arranged on inner circumferential surface 23a of band-shaped body 23 that forms belt 20 (see FIG. 2). Band-shaped body 23 is composed of a plastic material flexible in the thickness direction and non-stretchable in the circumferential direction (longitudinal direction).

Pressure cuff 24 is formed as a fluid bag that expands and contracts in the thickness direction of belt 20. The pressure cuff is in a pressurizing state by supply of fluid and in a non-pressurizing state by ejection of fluid. Pressure cuff 24 is formed as a fluid bag, for example, with two stretchable polyurethane sheets being opposed to each other in the thickness direction and with peripheral portions thereof being molten and joined.

Solid material 22 is arranged at a position corresponding to electrode group 40E on an inner circumferential surface 24a of pressure cuff 24. Solid material 22 is composed, for example, of a plate-shaped resin (for example, polypropylene) having a thickness approximately from 1 to 2 mm. In the present embodiment, belt 20, pressure cuff 24, and solid material 22 are used as a pressing portion that presses sensor unit 40 against a measurement site (a site corresponding to radial artery 91).

Sensor unit 40A is arranged and constructed similarly to sensor unit 40. Specifically, an electrode group 40F is arranged on inner circumferential surface 20a at a site where sensor unit 40A is arranged. Electrode group 40F includes six plate-shaped (or sheet-shaped) electrodes 41A to 46A arranged as being distant from one another in width direction Y of belt 20. A site where electrode group 40F is arranged corresponds to ulnar artery 91A in wrist 90 in the attached state.

A solid material 22A is arranged at a position corresponding to electrode group 40F on outer circumferential surface 21a. A pressure cuff 24A is arranged on an outer circumferential side of solid material 22A. Pressure cuff 24A is an expansion member that locally presses a region corresponding to electrode group 40F in the circumferential direction of compression cuff 21. Pressure cuff 24A is also arranged on inner circumferential surface 23a of band-shaped body 23 that forms belt 20, similarly to pressure cuff 24 (see FIG. 2).

Pressure cuff 24A is formed as a fluid bag that expands and contracts in the thickness direction of belt 20. The pressure cuff is in the pressurizing state by supply of fluid and in the non-pressurizing state by ejection of fluid. Pressure cuff 24A is formed as a fluid bag, for example, with two stretchable polyurethane sheets being opposed to each other in the thickness direction and with peripheral portions thereof being molten and joined.

Solid material 22A is arranged at a position corresponding to electrode group 40F on an inner circumferential surface 24b of pressure cuff 24A. Solid material 22A is composed, for example, of a plate-shaped resin (for example, polypropylene) having a thickness approximately from 1 to 2 mm. In the present embodiment, belt 20, pressure cuff 24A, and solid material 22A are used as a pressing portion that presses sensor unit 40A against a measurement site (a site corresponding to ulnar artery 91A).

As shown in FIG. 1, a bottom surface (a surface closest to a measurement site) 10b of main body 10 and end 20f of belt 20 are connected to each other by a Z-fold buckle 15 (which will also simply be referred to as a “buckle 15” below).

Buckle 15 includes a plate-shaped member 25 arranged on the outer circumferential side and a plate-shaped member 26 arranged on the inner circumferential side. Plate-shaped member 25 has one end 25e pivotably attached to main body 10 with a coupling rod 27 extending along width direction Y being interposed. Plate-shaped member 25 has the other end 25f pivotably attached to one end 26e of plate-shaped member 26 with a coupling rod 28 extending along width direction Y being interposed. Plate-shaped member 26 has the other end 26f fixed in the vicinity of end 20f of belt 20 by a fixing portion 29.

A position of attachment of fixing portion 29 in the circumferential direction of belt 20 is variably set in advance, in conformity with a length of a perimeter of wrist 90 of a user. Blood pressure monitor 1 (belt 20) is thus formed substantially annularly as a whole and bottom surface 10b of main body 10 and end 20f of belt 20 are opened and closed by means of buckle 15 in a direction shown with an arrow B in FIG. 1.

When a user attaches blood pressure monitor 1 to wrist 90, the user passes his/her left hand through a ring of belt 20 in a direction shown with an arrow A in FIG. 1 with buckle 15 being opened to increase a diameter of the ring of belt 20. Then, as shown in FIG. 2, the user adjusts an angular position of belt 20 around wrist 90 by sliding or the like and moves sensor unit 40 so as to be located above radial artery 91. Electrode group 40E of sensor unit 40 thus abuts on a part 90a1 of palm-side surface 90a of wrist 90 that corresponds to radial artery 91. Electrode group 40F of sensor unit 40A abuts on a part of palm-side surface 90a of wrist 90 that corresponds to ulnar artery 91A. The user fixes the blood pressure monitor by closing buckle 15 in this state. The user thus attaches blood pressure monitor 1 (belt 20) by wrapping the same around wrist 90.

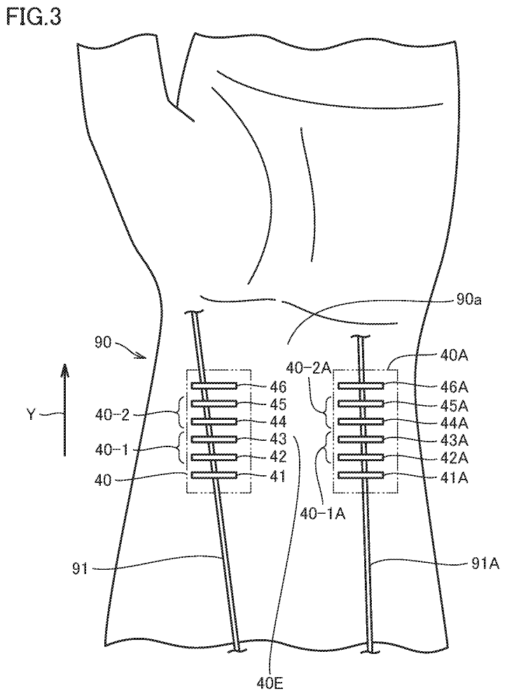

FIG. 3 is a diagram showing a two-dimensional layout of the electrode group for impedance measurement with blood pressure monitor 1 according to the first embodiment being attached to wrist 90. Referring to FIG. 3, in the attached state, electrode group 40E of sensor unit 40 is aligned along the longitudinal direction of the wrist in correspondence with radial artery 91 in left wrist 90. Electrode group 40E includes a pair of current electrodes 41 and 46 for current feed that is arranged on opposing sides in width direction Y and a pair of detection electrodes 42 and 43 and a pair of detection electrodes 44 and 45 arranged between the pair of current electrodes 41 and 46. A first pulse wave sensor 40-1 includes the pair of detection electrodes 42 and 43 and a second pulse wave sensor 40-2 includes the pair of detection electrodes 44 and 45.

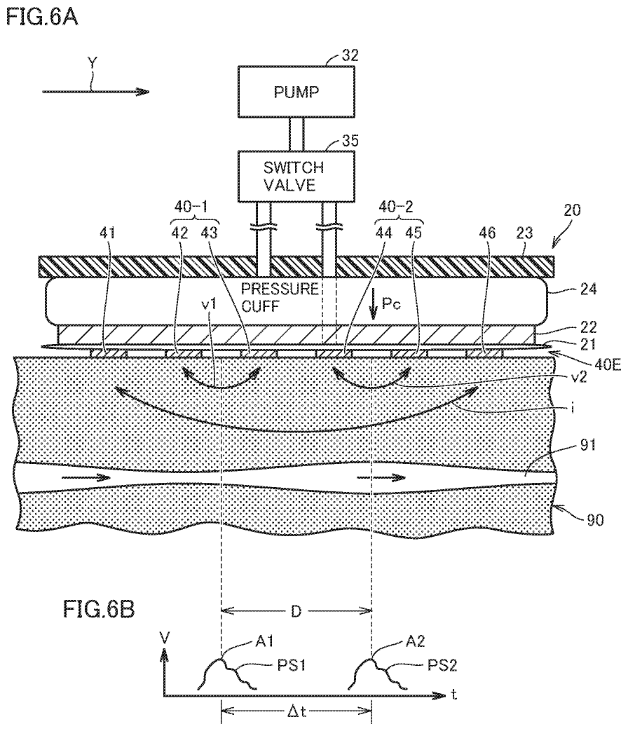

The pair of detection electrodes 44 and 45 is arranged in correspondence with a portion on a downstream side of bloodstream in radial artery 91, with respect to the pair of detection electrodes 42 and 43. In width direction Y, a distance D (see FIG. 6B which will be described later) between the center between the pair of detection electrodes 42 and 43 and the center between the pair of detection electrodes 44 and 45 is set, for example, to 20 mm. Distance D corresponds to a distance between first pulse wave sensor 40-1 and second pulse wave sensor 40-2. In width direction Y, a distance between the pair of detection electrodes 42 and 43 and a distance between the pair of detection electrodes 44 and 45 are each set, for example, to 2 mm.

Similarly, in the attached state, electrode group 40F of sensor unit 40A is aligned along the longitudinal direction of the wrist in correspondence with ulnar artery 91A in left wrist 90. Electrode group 40F includes a pair of current electrodes 41A and 46A for current feed arranged on opposing sides in width direction Y and a pair of detection electrodes 42A and 43A and a pair of detection electrodes 44A and 45A arranged between the pair of current electrodes 41A and 46A. A first pulse wave sensor 40-1A includes the pair of detection electrodes 42A and 43A and a second pulse wave sensor 40-2A includes the pair of detection electrodes 44A and 45A.

The pair of detection electrodes 44A and 45A is arranged in correspondence with a portion on a downstream side of bloodstream in ulnar artery 91A, with respect to the pair of detection electrodes 42A and 43A. In width direction Y, distance D between the center between the pair of detection electrode 42A and 43A and the center between the pair of detection electrodes 44A and 45A is set, for example, to 20 mm. Distance D corresponds to a distance between first pulse wave sensor 40-1A and second pulse wave sensor 40-2A. In width direction Y, a distance between the pair of detection electrodes 42A and 43A and a distance between the pair of detection electrodes 44A and 45A are each set, for example, to 2 mm.

Since electrode groups 40E and 40F can be formed to be low in profile, belt 20 as a whole can be formed to be small in thickness in blood pressure monitor 1. Since electrode groups 40E and 40F can be formed to be flexible, electrode groups 40E and 40F do not interfere with compression of left wrist 90 by compression cuff 21 and does not compromise accuracy in measurement of a blood pressure with an oscillometric method which will be described later.

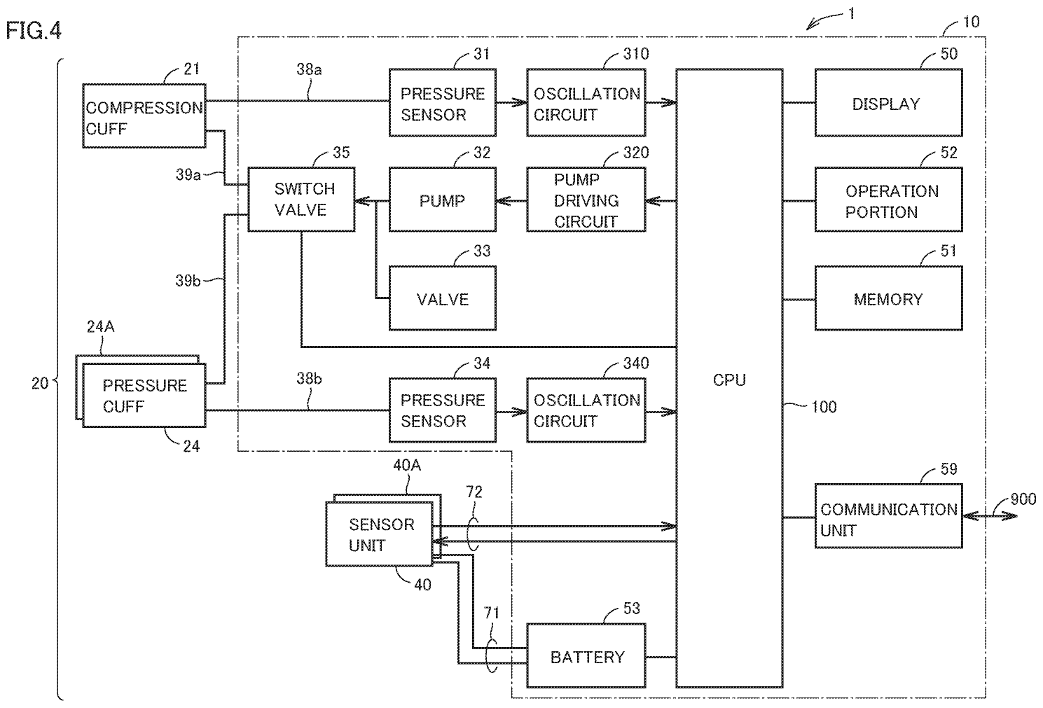

FIG. 4 is a diagram showing a block configuration of a control system of blood pressure monitor 1 according to the first embodiment. Blood pressure monitor 1 performs a function to measure a blood pressure with the oscillometric method and a function to measure a blood pressure based on a PTT. A configuration in which air is employed as fluid in blood pressure monitor 1 in FIG. 4 is illustrated.

Referring to FIG. 4, main body 10 includes a central processing unit (CPU) 100 that functions as control circuits, a display 50, a memory 51 that functions as a storage, an operation portion 52 as an operation circuit, a battery 53, and a communication unit 59. Main body 10 includes a pressure sensor 31, a pump 32, a valve 33, a pressure sensor 34, and a switch valve 35. Switch valve 35 switches a component to which pump 32 and valve 33 are to be connected, between compression cuff 21 and pressure cuffs 24 and 24A.

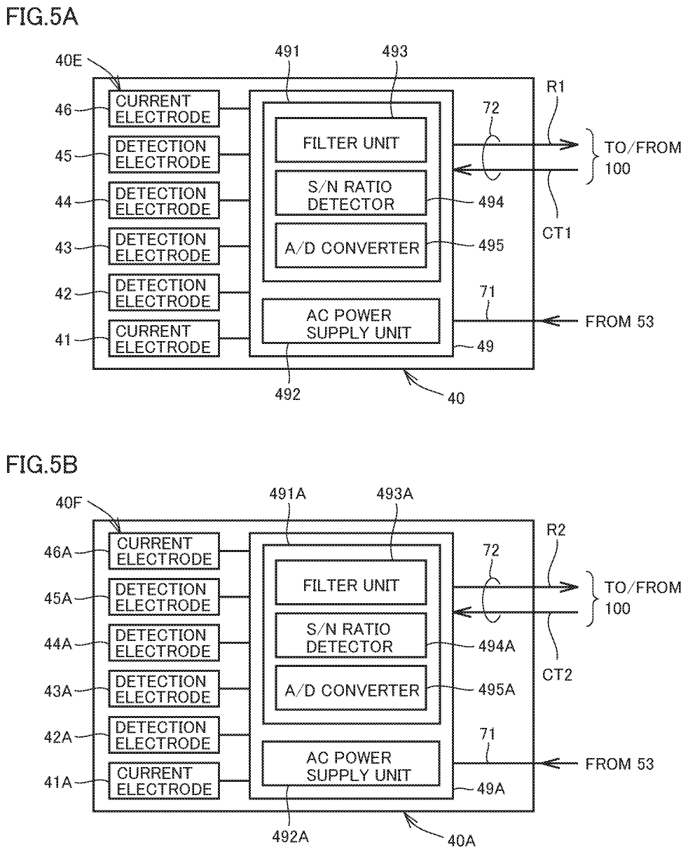

Main body 10 further includes an oscillation circuit 310 and an oscillation circuit 340 that convert outputs from pressure sensor 31 and pressure sensor 34 into a frequency and a pump driving circuit 320 that drives pump 32. A configuration of sensor units 40 and 40A will be described later with reference to FIGS. 5A and 5B.

Display 50 is implemented, for example, by an organic electro luminescence (EL) display and shows information in accordance with a control signal from CPU 100. This information includes a result of measurement. Display 50 is not limited to the organic EL display but may be implemented, for example, by a display of another type such as a liquid crystal display (LCD).

Operation portion 52 is implemented, for example, by a push switch circuit, and provides an operation signal in accordance with an instruction to start or stop measurement of a blood pressure by a user to CPU 100. Operation portion 52 is not limited to the push switch but may be implemented, for example, by a pressure-sensitive (resistive) or proximity (capacitive) touch panel switch. Alternatively, main body 10 may include a microphone (not shown) and accept an instruction to start measurement of a blood pressure through voice of a user.

Memory 51 stores in a non-transitory manner, data of a program for control of blood pressure monitor 1, data used for control of blood pressure monitor 1, setting data for setting of various functions of blood pressure monitor 1, and data on a result of measurement of a blood pressure value. Memory 51 is used as a work memory in execution of a program.

CPU 100 performs various functions as a control unit, in accordance with a program for control of blood pressure monitor 1 stored in memory 51. For example, in conducting measurement of a blood pressure with the oscillometric method, CPU 100 drives pump 32 (and valve 33) based on a signal from pressure sensor 31 in response to an instruction to start measurement of a blood pressure from operation portion 52. CPU 100 calculates a blood pressure value (a highest blood pressure (a systolic blood pressure) and a lowest blood pressure (a diastolic blood pressure)) based on a signal from pressure sensor 31 and calculates a pulse rate.

In conducting measurement of a blood pressure based on the PTT, CPU 100 drives valve 33 for ejecting air in compression cuff 21 in response to an instruction to start measurement of a blood pressure from operation portion 52. CPU 100 drives switch valve 35 to switch a component to which pump 32 (and valve 33) is to be connected, to pressure cuffs 24 and 24A. CPU 100 further calculates a blood pressure value based on a signal from pressure sensor 34.

Communication unit 59 is controlled by CPU 100 to communicate with an external information processing apparatus through a network 900. Though the external information processing apparatus may include a portable terminal 10B and a server 30 which will be described alter, it is not limited to such apparatuses. Communication through network 900 may include wireless or wired communication. For example, network 900 may include the Internet and a local area network (LAN). Alternatively, one-to-one communication through a USB cable may also be included in the communication through network 900. Communication unit 59 may include circuits such as a micro USB connector.

Pump 32 and valve 33 are connected to compression cuff 21 and pressure cuffs 24 and 24A with switch valve 35 and air pipes 39a and 39b being interposed. Pressure sensor 31 is connected to compression cuff 21 through an air pipe 38a and pressure sensor 34 is connected to pressure cuffs 24 and 24A through an air pipe 38b. Pressure sensor 31 detects a pressure in compression cuff 21 through air pipe 38a. Switch valve 35 is driven based on a control signal provided by CPU 100 and switches a component to which pump 32 and valve 33 are to be connected, between compression cuff 21 and pressure cuffs 24 and 24A.

Pump 32 is implemented, for example, by a piezoelectric pump. When switch valve 35 switches the component to which pump 32 and valve 33 are to be connected to compression cuff 21, pump 32 supplies air as pressurization fluid to compression cuff 21 through air pipe 39a for increase in pressure (cuff pressure) in compression cuff 21. When switch valve 35 switches the component to which pump 32 and valve 33 are to be connected to pressure cuffs 24 and 24A, pump 32 supplies air to pressure cuffs 24 and 24A through air pipe 39b for increase in pressure (cuff pressure) in pressure cuffs 24 and 24A.

Valve 33 is mounted on pump 32 and controlled to open and close in response to on/off of pump 32. Specifically, when switch valve 35 switches the component to which pump 32 and valve 33 are to be connected to compression cuff 21, with turn-on of pump 32, valve 33 is closed to seal air in compression cuff 21, whereas with turn-off of pump 32, valve 33 is opened to eject air in compression cuff 21 into the atmosphere through air pipe 39a.

When switch valve 35 switches the component to which pump 32 and valve 33 are to be connected to pressure cuffs 24 and 24A, with turn-on of pump 32, valve 33 is closed to seal air in pressure cuffs 24 and 24A, whereas with turn-off of pump 32, valve 33 is opened to eject air in pressure cuffs 24 and 24A into the atmosphere through air pipe 39b. Valve 33 performs a function as a check valve and backflow of ejected air does not occur. Pump driving circuit 320 drives pump 32 based on a control signal provided by CPU 100.

Pressure sensor 31 is implemented, for example, by a piezoresistive pressure sensor and connected to pump 32, valve 33, and compression cuff 21 through air pipe 38a. Pressure sensor 31 detects through air pipe 38a, a pressure applied by belt 20 (compression cuff 21) such as a pressure with an atmospheric pressure being defined as the reference (zero), and outputs the pressure as a time-series signal.

Oscillation circuit 310 outputs to CPU 100, a frequency signal having a frequency in accordance with a value of an electrical signal from pressure sensor 31 based on variation in electrical resistance owing to a piezoresistive effect. Output from pressure sensor 31 is used for control of a pressure applied by compression cuff 21 and calculation of a blood pressure value with the oscillometric method.

Pressure sensor 34 is implemented, for example, by a piezoresistive pressure sensor and connected to pump 32, valve 33, and pressure cuffs 24 and 24A through air pipe 38b. Pressure sensor 34 detects through air pipe 38b, a pressure applied by pressure cuffs 24 and 24A such as a pressure with an atmospheric pressure being defined as the reference (zero) and outputs the pressure as a time-series signal.

Oscillation circuit 340 oscillates in accordance with a value of an electrical signal from pressure sensor 34 based on variation in electrical resistance owing to a piezoresistive effect and outputs a frequency signal having a frequency in accordance with the value of the electrical signal from pressure sensor 34. Output from pressure sensor 34 is used for control of a pressure applied by pressure cuffs 24 and 24A and calculation of a blood pressure based on the PTT. In control of a pressure applied by pressure cuffs 24 and 24A for measurement of a blood pressure based on the PTT, CPU 100 controls pump 32 and valve 33 to increase and decrease a cuff pressure in accordance with various conditions. Battery 53 supplies electric power to various elements mounted on main body 10. Battery 53 supplies electric power also to sensor units 40 and 40A and portion 49 through a line 71. Line 71 is provided to extend between main body 10 and sensor units 40 and 40A along the circumferential direction of belt 20 with line 71, together with a signal line 72, lying between band-shaped body 23 of belt 20 and compression cuff 21.

(Configuration of Sensor Unit)

FIGS. 5A and 5B are diagrams showing a circuit configuration of the sensor unit according to the first embodiment. Referring to FIG. 5A, sensor unit 40 includes electrodes 41 to 46 in electrode group 40E described previously and a current feed and voltage detector 49 as circuits. Current feed and voltage detector 49 includes an alternating current (AC) power supply unit 492 (corresponding to a first output unit) that outputs a first current signal having a first frequency to a corresponding measurement site through current electrodes 41 and 46 and a voltage detector 491 (corresponding to a first detector) that detects with detection electrodes 42 to 45, a voltage signal representing pulse waves from the corresponding measurement site.

AC power supply unit 492 applies a voltage having the first frequency to current electrodes 41 and 46 by receiving a voltage from battery 53 in response to a control signal CT1 from CPU 100. A current is thus supplied to the measurement site. Voltage detector 491 detects a voltage signal from the measurement site with detection electrodes 42 to 45 in response to control signal CT1 from CPU 100. Voltage detector 491 includes a filter unit 493 that includes a band-pass filter (BPF) circuit having a filter characteristic (such as a cut-off frequency) corresponding to the first frequency, a signal-noise ratio (S/N ratio) detector 494 having circuits that detect an S/N ratio of the detected voltage signal, and an analog-digital (A/D) converter 495 having circuits that convert a voltage signal into digital data. Voltage detector 491 outputs a detected S/N ratio R1 and resultant digital data to CPU 100.

Referring to FIG. 5B, sensor unit 40A includes electrodes 41A to 46A in electrode group 40F described previously and a current feed and voltage detector 49A, as circuits. Current feed and voltage detector 49A includes an AC power supply unit 492A (corresponding to a second output unit) that outputs a second current signal having a second frequency to a corresponding measurement site through current electrodes 41A and 46A and a voltage detector 491A (corresponding to a second detector) that detects with detection electrodes 42A to 45A, a voltage signal representing pulse waves from the corresponding measurement site.

AC power supply unit 492A applies a voltage having the second frequency to current electrodes 41A and 46A by receiving a voltage from battery 53 in response to a control signal CT2 from CPU 100. A current is thus supplied to the measurement site. Voltage detector 491A detects a voltage signal from the measurement site with detection electrodes 42A to 45A in response to control signal CT2 from CPU 100. Voltage detector 491A includes a filter unit 493A including a BPF circuit having a filter characteristic (a cut-off frequency) corresponding to the second frequency, a signal-noise ratio (S/N ratio) detector 494A having circuits that detect an S/N ratio of the detected voltage signal, and an A/D converter 495A having circuits that convert a voltage signal into digital data. Voltage detector 491A outputs a detected S/N ratio R2 and resultant digital data to CPU 100.

AC power supply units 492 and 492A may include a boost circuit and a voltage regulation circuit that generate voltage signals having the first frequency and the second frequency upon receiving a voltage from battery 53.

(Overview of Measurement of Blood Pressure Based on Pulse Wave Transit Time)

FIGS. 6A and 6B are schematic diagrams for illustrating measurement of a blood pressure based on a pulse wave transit time according to the first embodiment. Specifically, FIG. 6A shows a schematic cross-section along the longitudinal direction of the wrist in measurement of a blood pressure based on a pulse wave transit time with blood pressure monitor 1 being attached to wrist 90. FIG. 6B shows a waveform of pulse wave signals PS1 and PS2. Though FIGS. 6A and 6B show a state that sensor unit 40 is located above radial artery 91 at the measurement site, description the same as description with reference to FIGS. 6A and 6B is applicable also to a state that sensor unit 40A is located above ulnar artery 91A at the measurement site. Therefore, measurement of a blood pressure based on a pulse wave transit time by sensor unit 40A is briefly described.

Referring to FIG. 6A, AC power supply unit 492 feeds, for example, a high-frequency constant current i at a current value of 1 mA having the first frequency to the measurement site by applying a prescribed voltage across the pair of current electrodes 41 and 46.

Voltage detector 491 detects a voltage signal v1 across the pair of detection electrodes 42 and 43 implementing first pulse wave sensor 40-1 and a voltage signal v2 across the pair of detection electrodes 44 and 45 implementing second pulse wave sensor 40-2. Voltage signals v1 and v2 represent variation in electrical impedance caused by pulse waves from bloodstream in radial artery 91 in respective portions in palm-side surface 90a of left wrist 90 to which first pulse wave sensor 40-1 and second pulse wave sensor 40-2 are opposed.

Specifically, in voltage detector 491, a component except for a signal component corresponding to the first frequency is removed by filter unit 493 from voltage signals v1 and v2. S/N ratio detector 494 detects the S/N ratio of the voltage signal that has passed through the filter. A/D converter 495 converts voltage signals v1 and v2 that have passed through filter unit 493 from analog data to digital data and outputs the digital data to CPU 100 through line 72.

CPU 100 subjects input voltage signals v1 and v2 (digital data) to prescribed signal processing and generates pulse wave signals PS1 and PS2 having a waveform like a crest as shown in FIG. 6B.

Voltage signals v1 and v2 are around, for example, 1 mv. Pulse wave signals PS1 and PS2 have respective peaks A1 and A2, for example, around 1 V. When a pulse wave velocity (PWV) of bloodstream in radial artery 91 is assumed to be within a range from 1000 cm/s to 2000 cm/s, a time interval Δt between pulse wave signal PS1 and pulse wave signal PS2 is within a range from 1.0 ms to 2.0 ms because distance D between first pulse wave sensor 40-1 and second pulse wave sensor 40-2 is D=20 mm.

Sensor unit 40A also feeds current to the measurement site of above ulnar artery 91. Specifically, AC power supply unit 492A of sensor unit 40A feeds high-frequency constant current i, for example, at a current value of 1 mA having the second frequency to the measurement site by applying a prescribed voltage across the pair of current electrodes 41A and 46A.

Voltage detector 491A detects a voltage signal v1A across the pair of detection electrodes 42A and 43A implementing first pulse wave sensor 40-1A and a voltage signal v2A across the pair of detection electrodes 44A and 45A implementing second pulse wave sensor 40-2A. Voltages signals v1A and v2A represent variation in electrical impedance caused by pulse waves of bloodstream in ulnar artery 91A in respective portions of palm-side surface 90a of left wrist 90 to which first pulse wave sensor 40-1A and second pulse wave sensor 40-2A are opposed.

In voltage detector 491A, a component except for a signal component corresponding to the second frequency is removed by filter unit 493A from voltage signals v1A and v2A. S/N ratio detector 494A detects the S/N ratio of the voltage signal that has passed through the filter. A/D converter 495A converts voltage signals v1 and v2 that have passed through filter unit 493A from analog data to digital data and outputs the digital data to CPU 100 through line 72. Though a sampling rate of A/D converter 495 and A/D converter 495A is set, for example, to 300 Hz, the sampling rate is not limited to this rate and a sampling rate necessary for maintaining accuracy in calculation based on the PTT should only be set.

CPU 100 subjects input voltage signals v1A and v2A (digital data) to prescribed signal processing and generates pulse wave signals PS1A and PS2A. Distance D and time interval Δt between peaks A1 and A2 of respective pulse wave signals PS1A and PS2A are detected similarly as described above.

As shown in FIG. 6A, pressure cuff 24 is in the pressurizing state and compression cuff 21 is in the non-pressurizing state with air therein having been ejected. Pressure cuff 24 and solid material 22 are arranged across first pulse wave sensor 40-1, second pulse wave sensor 40-2, and the pair of current electrodes 41 and 46 in the direction in which radial artery 91 extends. Therefore, when a pressure is applied by pump 32, pressure cuff 24 presses first pulse wave sensor 40-1, second pulse wave sensor 40-2, and the pair of current electrodes 41 and 46 against palm-side surface 90a of wrist 90 with solid material 22 being interposed.

Force with which the pair of current electrodes 41 and 46, first pulse wave sensor 40-1, and second pulse wave sensor 40-2 are pressed against palm-side surface 90a of wrist 90 can be set to an appropriate value. Since pressure cuff 24 in the form of the fluid bag is employed as the pressing portion in the present embodiment, pump 32 and valve 33 can be used in common to compression cuff 21 so that the configuration can be simplified. Since first pulse wave sensor 40-1, second pulse wave sensor 40-2, and the pair of current electrodes 41 and 46 can be pressed with solid material 22 being interposed, force of pressing against the measurement site is uniform and a blood pressure can accurately be measured based on a pulse wave transit time. Such a feature can similarly be achieved also when measurement is conducted with the use of sensor unit 40A.

(Functional Configuration of CPU 100)

FIG. 8 is a diagram schematically showing a configuration of a function relating to measurement provided by CPU 100 according to the first embodiment. Referring to FIG. 8, CPU 100 includes a blood pressure calculator 110 that calculates (estimates) a blood pressure, a display controller 120 that controls display 50, a memory controller 130 that controls writing of data into memory 51 or reading of data from memory 51, and a communication controller 140 that controls communication unit 59.

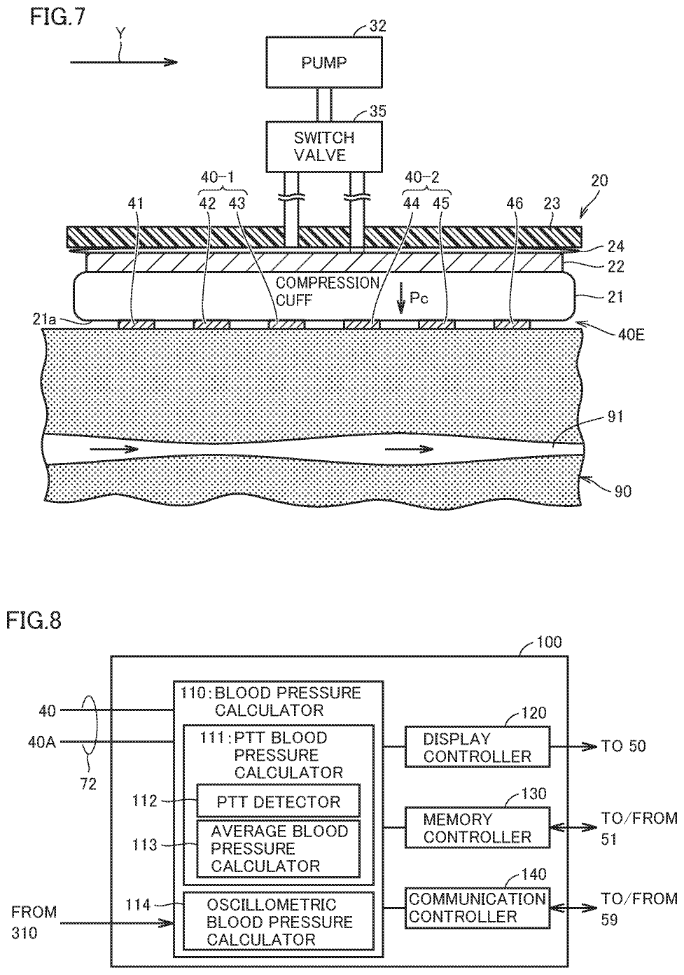

Blood pressure calculator 110 includes a PTT blood pressure calculator 111 corresponding to the function to measure a blood pressure based on a PTT and an oscillometric blood pressure calculator 114 corresponding to the function to measure a blood pressure in accordance with the oscillometric method shown in FIG. 7. PTT blood pressure calculator 111 includes a PTT detector 112 and an average blood pressure calculator 113. Details of the function of each component will be described later.

(Operation to Measure Blood Pressure Based on PTT)

The function to measure a blood pressure based on the PTT by PTT blood pressure calculator 111 will be described. Initially, when a user indicates measurement of a blood pressure based on a PTT through operation portion 52, CPU 100 starts up PTT blood pressure calculator 111. CPU 100 drives switch valve 35 in response to the instruction from the user and switches a component to which pump 32 and valve 33 are to be connected, to pressure cuffs 24 and 24A. Thereafter, CPU 100 closes valve 33, drives pump 32 by means of pump driving circuit 320 to send air to pressure cuffs 24 and 24A, and increases a cuff pressure Pc representing a pressure in pressure cuffs 24 and 24A at a constant rate.

In this pressurization process, PTT detector 112 of CPU 100 obtains first and second pulse wave signals PS1 and PS2 output on a time-series basis from first pulse wave sensor 40-1 and second pulse wave sensor 40-2 of sensor unit 40 and calculates in real time, a cross-correlation coefficient r between waveforms of first and second pulse wave signals PS1 and PS2. When CPU 100 determines that cross-correlation coefficient r calculated in real time in the pressurization process exceeds a threshold value Th (for example, Th=0.99), it calculates as a pulse wave transit time (PTT), time interval Δt between peaks A1 and A2 of amplitudes of first and second pulse wave signals PS1 and PS2 detected at cuff pressure Pc at that time point.

Similarly, in this pressurization process, PTT detector 112 of CPU 100 obtains first and second pulse wave signals PS1A and PS2A from first pulse wave sensor 40-1A and second pulse wave sensor 40-2A of sensor unit 40A and calculates cross-correlation coefficient r between waveforms of these pulse wave signals. When CPU 100 determines that cross-correlation coefficient r calculated in real time in the pressurization process exceeds threshold value Th, it calculates as the pulse wave transit time (PTT), time interval Δt between peaks of amplitudes of first and second pulse wave signals PS1A and PS2A detected at cuff pressure Pc at that time point.

PTT blood pressure calculator 111 of CPU 100 calculates (estimates) a blood pressure EBP based on the PTT in accordance with outputs from sensor units 40 and 40A under a known expression (EBP=(α/(DT²)+β)). α and β in this expression are prescribed coefficients and DT represents a pulse wave transit time. Thus, blood pressure EBP based on the PTT of radial artery 91 (which is also referred to as a blood pressure EBP-1 below) and blood pressure EBP based on the PTT of ulnar artery 91A (which is also referred to as EBP-2 below) are measured. Average blood pressure calculator 113 calculates an average of blood pressure EBP-1 and blood pressure EBP-2.

CPU 100 repeatedly calculates the PTT and blood pressure EBP while an instruction to stop measurement is not given after an instruction to start measurement is given through operation portion 52. When CPU 100 receives an instruction to stop measurement through operation portion 52, it controls each component to quit the measurement operation.

(Overview of Measurement of Blood Pressure with Oscillometric Method)

The function to measure a blood pressure in accordance with the oscillometric method with oscillometric blood pressure calculator 114 will be described. Initially, when a user indicates oscillometric blood pressure measurement through operation portion 52, CPU 100 starts up oscillometric blood pressure calculator 114. FIG. 7 is a schematic cross-sectional view along the longitudinal direction of wrist 90 with blood pressure monitor 1 being attached to the wrist in measurement of a blood pressure with the oscillometric method according to the first embodiment.

Referring to FIG. 7, pressure cuff 24 is in the non-pressurizing state with air therein having been ejected and compression cuff 21 is in the pressurizing state with air being supplied thereto. Compression cuff 21 extends in the circumferential direction of wrist 90. When a pressure is applied by pump 32, compression cuff 21 evenly compresses left wrist 90 in the circumferential direction. Since only electrode group 40E is present between the inner circumferential surface of compression cuff 21 and left wrist 90, compression by compression cuff 21 is not blocked by other members and blood vessels can sufficiently be compressed.

In blood pressure measurement with the oscillometric method, oscillometric blood pressure calculator 114 calculates (estimates) a blood pressure in accordance with a waveform output from first pressure sensor 31 through oscillation circuit 310 and detected in the process of pressurization or reduction in pressure of the measurement site by compression cuff 21. Since a method of calculating a blood pressure with the oscillometric method according to the present embodiment follows a known method, description will not be repeated here.

Display controller 120 generates representation data based on various types of information including a blood pressure calculated by blood pressure calculator 110 and drives display 50 in accordance with the generated representation data. Display 50 thus shows information including the measured blood pressure. Memory controller 130 has memory 51 store various types of information including the blood pressure calculated by blood pressure calculator 110. Memory 51 can thus save a history of information including the measured blood pressure. Memory controller 130 reads various types of information including the blood pressure calculated by blood pressure calculator 110 from memory 51. Communication controller 140 transmits various types of information including the blood pressure calculated by blood pressure calculator 110 or read from memory 51 through communication unit 59 to an external information processing apparatus and has the information processing apparatus show the information.

The function of each component in FIG. 8 is stored as a program in memory 51. CPU 100 performs the function of each component by reading a program from memory 51 and executing the program. The function of each component is not limited to the function implemented by the program. The function may be performed, for example, by circuitry including an application specific integrated circuit (ASIC) or a field-programmable gate array (FPGA). Furthermore, the function may be implemented by combination of a program and circuitry. The program may be executed by at least one hardware processor such as CPU 20 or combination of a processor and a circuit such as an ASIC or an FPGA.

(Flowchart of Processing)

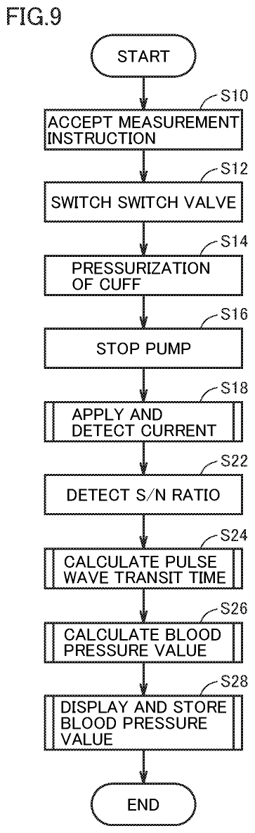

FIG. 9 is a flowchart showing processing in measurement of a blood pressure based on the PTT according to the first embodiment. A program in accordance with the flowchart is stored in memory 51 and read and executed by CPU 100.

Referring to FIG. 9, initially, when a user performs a switch operation onto operation portion 52 to start measurement of a blood pressure based on the PTT in the attached state, CPU 100 accepts the start instruction (step S10). In starting measurement of a blood pressure, CPU 100 controls switch valve 35 to switch the component to which pump 32 and valve 33 are to be connected to pressure cuffs 24 and 24A (step S12). Air is thus exhausted from cuffs 24 and 24A.

CPU 100 drives pump 32 to pressurize pressure cuffs 24 and 24A to a prescribed pressure, and thereafter closes valve 33 (step S14) and thereafter stops pump 32 (step S16). CPU 100 outputs a current signal to a measurement site and outputs control signals CT1 and CT2 to sensor units 40 and 40A to detect a voltage signal representing pulse waves (step S18).

Sensor unit 40 outputs digital data of a voltage signal (pulse wave signal) detected at the measurement site corresponding to radial artery 91, detects S/N ratio R1 of a component of the first frequency in the voltage signal, and outputs the S/N ratio to CPU 100 (step S22). Similarly, sensor unit 40A outputs digital data of a voltage signal (pulse wave signal) detected at the measurement site corresponding to ulnar artery 91A, detects S/N ratio R2 of a component of the second frequency in the voltage signal, and outputs the S/N ratio to CPU 100 (step S22).

PTT detector 112 calculates the PTT in accordance with the pulse wave signals from sensor units 40 and 40A (step S24). PTT blood pressure calculator 111 calculates blood pressure EBP-1 based on the PTT corresponding to sensor unit 40 and calculates blood pressure EBP-2 based on the PTT corresponding to sensor unit 40A (step S26).

CPU 100 outputs blood pressure information based on calculated blood pressures EBP-1 and EBP-2 (step S28). For example, display controller 120 controls display 50 to show the blood pressure information. Alternatively, memory controller 130 has memory 51 store the blood pressure information. Alternatively, communication controller 140 transmits the blood pressure information to an external information processing apparatus through communication unit 59.

(Exemplary Storage of Result of Measurement)

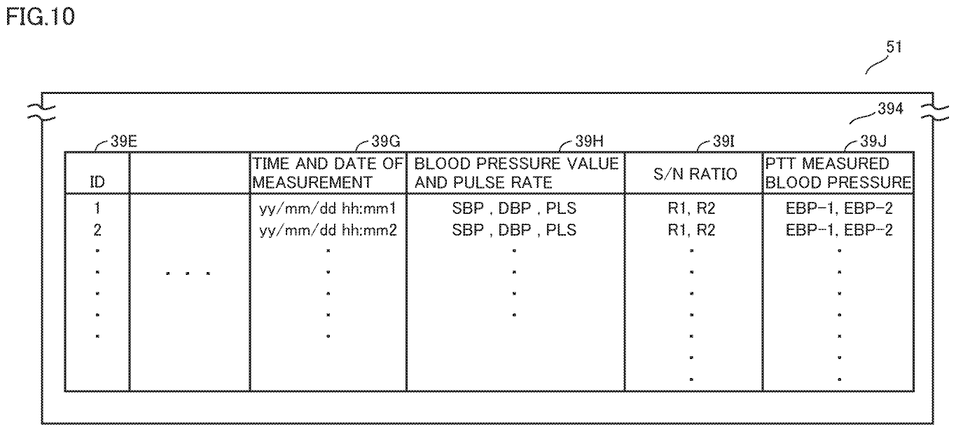

FIG. 10 is a diagram showing exemplary storage of a result of measurement according to the first embodiment. Referring to FIG. 10, memory 51 stores a table 394 that records a result of measurement by blood pressure monitor 1. Referring to FIG. 10, table 394 stores data on the result of measurement for each record unit. Each record includes in association, data 39E on identification (ID) for uniquely identifying the record, data 39G on time and date of measurement, data 39H including a blood pressure value (systolic blood pressure SBP and diastolic blood pressure DBP) and pulse rate PLS calculated (estimated) by oscillometric blood pressure calculator 114, S/N ratio data 391, and data 39J representing a blood pressure calculated (estimated) by PTT blood pressure calculator 111.

S/N ratio data 391 includes S/N ratio R1 detected for associated blood pressure EBP-1 and S/N ratio R2 detected for associated blood pressure EBP-2.

Data 39J includes blood pressure EBP-1 and blood pressure EBP-2 calculated (estimated) at the time of measurement of a blood pressure based on the PTT. Data 39J may further include a representative blood pressure EBP-R. Representative blood pressure EBP-R represents corresponding blood pressure EBP-1 and blood pressure EBP-2.

Memory controller 130 has memory 51 store in table 394 in association with data 39G on time and date of measurement, data 39H on a blood pressure and a pulse rate in accordance with the oscillometric method measured at that time and date and data 39J on the blood pressure value based on the PTT.

A manner of storage of measurement data in table 394 is not limited to storage by a record unit as shown in FIG. 10. Detected data 39E to 39J should only be associated (linked) with one another each time a blood pressure is measured.

(Method of Determining Representative Blood Pressure EBP-R)

Though representative blood pressure EBP-R is represented as an average blood pressure calculated by average blood pressure calculator 113 based on corresponding blood pressure EBP-1 and blood pressure EBP-2 in the first embodiment, representative blood pressure EBP-R is not limited to the average blood pressure.

For example, CPU 100 may determine a blood pressure that satisfies a predetermined condition, of blood pressure EBP-1 and blood pressure EBP-2, as representative blood pressure EBP-R. Under the predetermined condition, for example, a blood pressure larger (or smaller) in value, of blood pressure EBP-1 and blood pressure EBP-2, is determined as representative blood pressure EBP-R. Alternatively, a blood pressure that exceeds (or is equal to or smaller than) the threshold value, of blood pressure EBP-1 and blood pressure EBP-2, is determined as representative blood pressure EBP-R. Alternatively, a blood pressure higher in corresponding S/N ratio (lower in noise), of blood pressure EBP-1 and blood pressure EBP-2, is determined as representative blood pressure EBP-R. Alternatively, a blood pressure of which corresponding S/N ratio is larger (higher) than a predetermined threshold value, of blood pressure EBP-1 and blood pressure EBP-2, is determined as representative blood pressure EBP-R.

Average blood pressure calculator 113 performs a weighted average calculation function to calculate an average by weighting blood pressure EBP-1 and blood pressure EBP-2. Specifically, a weight for blood pressure EBP-1 is based on a value of corresponding S/N ratio R1 and a weight for blood pressure EBP-2 is based on corresponding S/N ratio R2. Average blood pressure calculator 113 sets the weight to be larger as the corresponding S/N ratio is higher (that is, noise is lower). Therefore, representative blood pressure EBP-R calculated based on a weighted average can express a value closer to a blood pressure higher in S/N ratio, of blood pressure EBP-1 and blood pressure EBP-2.

(Exemplary Representation)

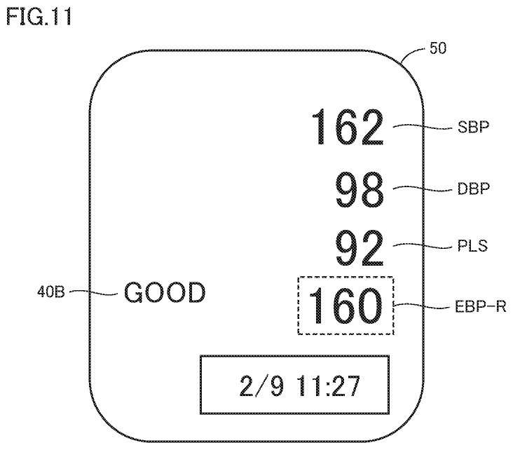

FIG. 11 is a diagram showing exemplary representation of a result of measurement according to the first embodiment. Referring to FIG. 11, a screen of display 50 includes systolic blood pressure SBP, diastolic blood pressure DBP, and pulse rate PLS based on the oscillometric method, representative blood pressure EBP-R, reliability 40B, and a date of measurement. Reliability 40B is based on a value of the S/N ratio corresponding to blood pressure EBP-1 and blood pressure EBP-2 on which representative blood pressure EBP-R is based. Reliability 40B includes reliability (or authenticity) of a value of shown representative blood pressure EBP-R.

In the first embodiment, reliability 40B can be based on S/N ratio R1 and S/N ratio R2 corresponding to blood pressure EBP-1 and blood pressure EBP-2 on which representative blood pressure EBP-R being shown on the same screen is based. For example, when CPU 100 determines that S/N ratio R1 and S/N ratio R2 have values larger (higher) than the threshold value, CPU 100 determines that reliability is high and shows reliability 40B with characters “GOOD” (see FIG. 11). In contrast, when CPU 100 determines that at least one of S/N ratio R1 and S/N ratio R2 has a value equal to or smaller than the threshold value, CPU 100 determines that reliability is low and shows reliability 40B with characters “NG”.

A manner of output of reliability is not limited to representation with such characters. For example, as to the manner of output, an image (picture) may be shown or a value of representative blood pressure EBP-R may be colored.

According to the screen in FIG. 11, a user can also obtain based on reliability 40B, a guideline as to whether or not shown blood pressure EBP-R is reliable.

Exemplary representation in FIG. 11 corresponds, for example, to exemplary representation at the time when measurement of a blood pressure ends (step S28) or exemplary representation of data read from table 394 in FIG. 10. Information in FIG. 11 is shown under control of display 50 by display controller 120. Specifically, display controller 120 generates representation data based on representative blood pressure EBP-R based on blood pressures EBP-1 and EBP-2 calculated by PTT blood pressure calculator 111, a value of the blood pressure calculated by oscillometric blood pressure calculator 114, and reliability 40B, and drives display 50 based on the representation data. Alternatively, display controller 120 generates representation data based on data 39H and data 39J associated in table 394 in FIG. 10 and reliability 40B, and drives display 50 based on the generated representation data. Display controller 120 can thus have display 50 show data on the measured blood pressure or data on the blood pressure stored in table 394.

(Configuration of System)

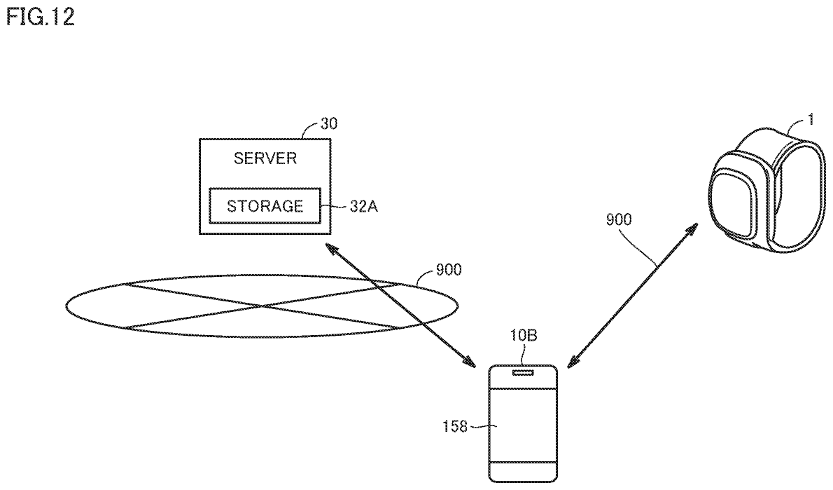

FIG. 12 is a diagram showing a schematic configuration of a system according to the first embodiment. Blood pressure monitor 1 communicates with server 30 or portable terminal 10B representing an external information processing apparatus through network 900. In the system in FIG. 12, blood pressure monitor 1 communicates with portable terminal 10B through a LAN and portable terminal 10B communicates with server 30 through the Internet. Blood pressure monitor 1 can thus communicate with server 30 via portable terminal 10B. Blood pressure monitor 1 may communicate with server 30 not via portable terminal 10B.

Though information in FIG. 11 is shown on display 50 of blood pressure monitor 1 in the first embodiment, CPU 100 may transmit the information to portable terminal 10B for representation on a display unit 158.

A location where a result of measurement shown in table 394 in FIG. 10 is stored is not limited to memory 51 of blood pressure monitor 1. For example, the result may be stored in a storage of portable terminal 10B or a storage 32A of server 30. Alternatively, the result may be stored in at least two of memory 51, the storage of portable terminal 10B, and storage 32A of server 30.

(Advantages of First Embodiment)

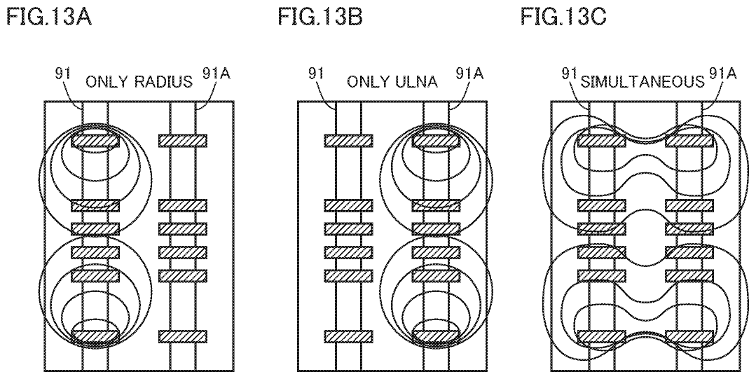

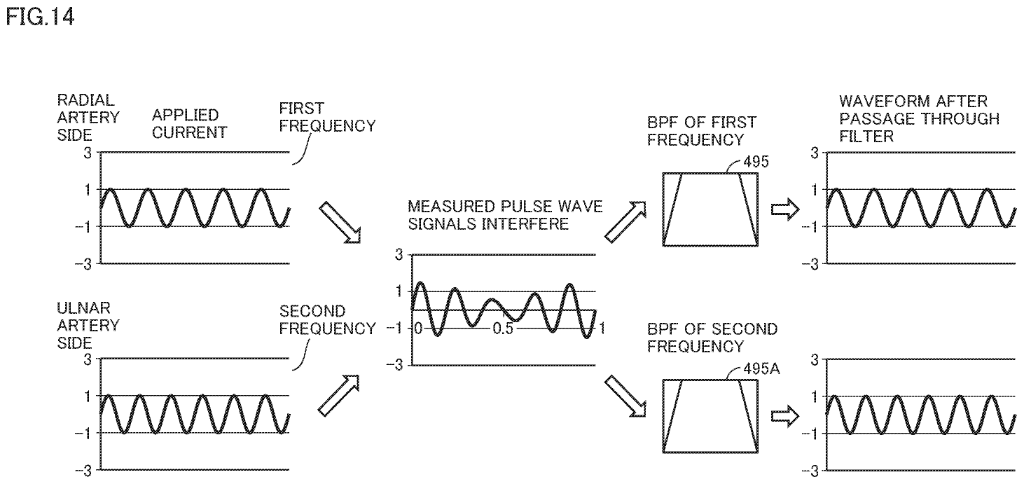

FIGS. 13A, 13B and 13C are diagrams for illustrating backgrounds of the first embodiment. FIG. 14 is a diagram showing a configuration of the first embodiment. Initially, when there are a plurality of sites as sites of measurement of pulse wave signals, in calculating a PTT based on an impedance, sites where a voltage signal (pulse wave signal) high in S/N ratio can be detected are varied depending on individual variation or a manner of attachment of blood pressure monitor 1. Therefore, desirably, a site where a voltage signal (pulse wave signal) high in S/N ratio can be detected is determined from among a plurality of measurement sites and a pulse wave signal is detected at the determined site.

When a current is simultaneously fed to measurement sites corresponding to both of radial artery 91 and ulnar artery 91A against such backgrounds, the currents may interfere with each other as shown in FIG. 13C and a potential distribution may be different from a normally obtained distribution.

In this connection, in the first embodiment, electrodes are arranged with both of radial artery 91 and ulnar artery 91A being designated as measurement sites as shown in FIG. 14, currents different in frequency (the first frequency or the second frequency) are output to the measurement sites, and a voltage signal representing pulse waves detected at each measurement site is processed based on a filter characteristic corresponding to the corresponding frequency.

Thus, even though interference occurs, a pulse wave signal free from a signal component resulting from interference can be extracted.

Furthermore, in the first embodiment, by selecting a pulse wave signal higher in S/N ratio described above, highly accurate pulse wave information and representative blood pressure EBP-R can be obtained.

(First Frequency and Second Frequency)

In the first embodiment, the first frequency is different from the second frequency in value. For example, one of 50 kHz and 60 kHz is defined as the first frequency and the other thereof is defined as the second frequency. A value of the first frequency and the second frequency, however, is not limited as such.

### Second Embodiment

Unlike the first embodiment, in a second embodiment, sensor unit 40 corresponding to the first pulse wave sensor unit and sensor unit 40A corresponding to the second pulse wave sensor unit are not simultaneously driven but alternately driven at predetermined intervals.

Blood pressure monitor 1 according to the second embodiment includes a CPU 100A that performs a function different from the function of CPU 100 in the first embodiment. Since the configuration of blood pressure monitor 1 according to the second embodiment is similar to the configuration shown in FIG. 1, description will not be repeated.

FIG. 15 is a diagram schematically showing a configuration of a function relating to measurement provided by CPU 100A according to the second embodiment. Referring to FIG. 15, CPU 100A includes a switching unit 150 in addition to the configuration of CPU 100 shown in FIG. 8. Since CPU 100A is similar in other functions to the CPU shown in FIG. 8, description will not be repeated.

Switching unit 150 outputs control signal CT1 to sensor unit 40 and outputs control signal CT2 to sensor unit 40A. Switching unit 150 alternately outputs control signal CT1 and control signal CT2 in predetermined cycles (at predetermined intervals) CR. Sensor unit 40 is driven while switching unit 150 outputs control signal CT1 and it is turned off while control signal CT1 is not output. Similarly, sensor unit 40A is driven while switching unit 150 outputs control signal CT2 and it is turned off while control signal CT2 is not output. Sensor unit 40 and sensor unit 40A operate as in the first embodiment while they are driven.

In the second embodiment, the first frequency of the first current signal output to a measurement site (a site corresponding to radial artery 91) by AC power supply unit 492 of sensor unit 40 and the second frequency of the second current signal output to a measurement site (a site corresponding to ulnar artery 91A) by AC power supply unit 492A of sensor unit 40A are equal to each other and set, for example, to 50 kHz, although they are not limited thereto. Therefore, filter unit 493 and filter unit 493A also have a frequency characteristic (cut-off frequency) in accordance with 50 kHz.

In the second embodiment, when a frequency of a current output to a measurement site is set to 50 kHz and a sampling rate for calculation based on the PTT is set, for example, to 300 Hz, a frequency of an output current is sufficiently high and hence cycle CR is set to a cycle corresponding to several hundred Hz to several kHz. This cycle is desirably determined based on a frequency of a current output to a measurement site and a sampling rate.

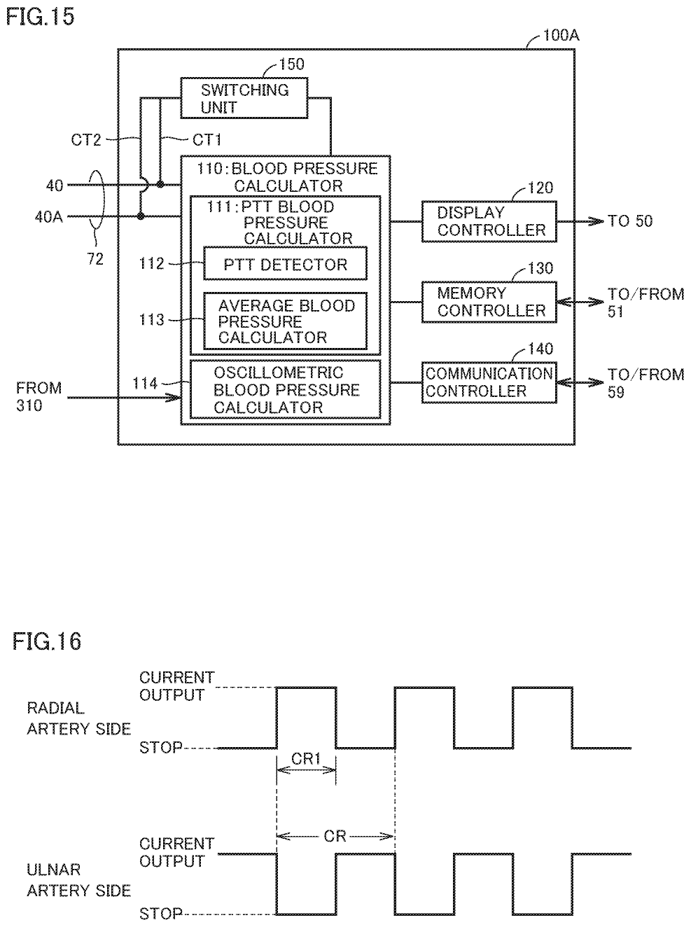

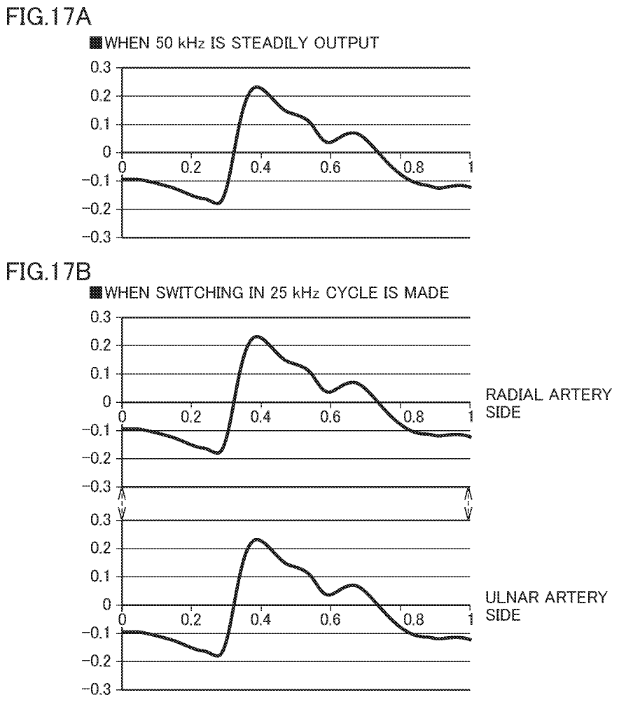

FIG. 16 is a diagram schematically showing cycle CR according to the second embodiment. Switching unit 150 alternately outputs control signal CT1 and control signal CT2 in cycles CR as shown in FIG. 16. Sensor unit 40 and sensor unit 40A are thus alternately driven for each half cycle CR1. FIGS. 17A and 17B are diagrams schematically showing a waveform of a current signal output to a measurement site according to the second embodiment. When a current signal at 50 kHz shown in FIG. 17A is output from sensor unit 40 or sensor unit 40A to a corresponding measurement site, switching unit 150 alternately outputs control signal CT1 and control signal CT2 in cycles CR in accordance with 25 kHz. At this time, waveforms (FIG. 17B) of a current signal output to the measurement site corresponding to radial artery 91 from sensor unit 40 and a current signal output to the measurement site corresponding to ulnar artery 91A from sensor unit 40A are similar to the waveform in FIG. 17A.

Processing for blood pressure measurement based on the PTT is performed in accordance with the flowchart shown in FIG. 9 also in the second embodiment.

(Advantages of Second Embodiment)

As described above, in calculating a PTT based on an impedance, sites where a voltage signal (pulse wave signal) high in S/N ratio can be detected are varied depending on individual variation or a manner of attachment of blood pressure monitor 1. Therefore, desirably, a site where a voltage signal (pulse wave signal) high in S/N ratio can be detected is determined from among a plurality of measurement sites and a pulse wave signal is detected at the determined site.

When a current is simultaneously fed to measurement sites corresponding to radial artery 91 and ulnar artery 91A against such backgrounds, the currents may interfere with each other as shown in FIG. 13C and a potential distribution may be different from a normally obtained distribution.

In this connection, in the second embodiment, switching unit 150 alternately outputs current signals equal in frequency at predetermined intervals (intervals in accordance with cycle CR) to measurement sites corresponding to radial artery 91 and ulnar artery 91A as shown in FIG. 16 and obtains information on pulse waves including the PTT from voltage signals representing pulse waves and detected at the measurement sites. Thus, when a current signal is output to one measurement site, the current signal is not output to the other measurement site as shown in FIG. 13A or FIG. 13B, and hence interference shown in FIG. 13C can be prevented from occurring.

By selecting a pulse wave signal higher in SN ratio also in the second embodiment as in the first embodiment, highly accurate pulse wave information and representative blood pressure EBP-R can also be obtained. A result of measurement is shown on display 50, stored in memory 51, and transmitted to an external information processing apparatus also in the second embodiment as in the first embodiment.

Though the first frequency and the second frequency are equal to each other in the second embodiment, they may be different from each other. For example, one of 50 kHz and 60 kHz is defined as the first frequency and the other thereof is defined as the second frequency as in the first embodiment.

### Third Embodiment

In a third embodiment, an operation mode of blood pressure monitor 1 will be described. Blood pressure monitor 1 includes as modes for measuring pulse wave information, a first mode and a second mode that are selectively started up. In the first mode, sensor unit 40 outputs a first current signal having the first frequency to a measurement site corresponding to radial artery 91 and processes a voltage signal representing a pulse wave signal and detected at the measurement site based on a filter characteristic corresponding to the first frequency. Sensor unit 40A outputs a second current signal having the second frequency to a measurement site corresponding to ulnar artery 91A at the time when sensor unit 40 outputs the first current signal and processes a voltage signal representing a pulse wave signal and detected at the measurement site based on a filter characteristic corresponding to the second frequency. In the first mode, switching unit 150 is off.

In the second mode, the first pulse wave sensor unit and the second pulse wave sensor unit are alternately driven at predetermined intervals by switching unit 150.

In any of the first mode and the second mode, blood pressure monitor 1 can obtain information on pulse waves including the PTT, without being affected by interference described above.

A user can instruct CPU 100 to start up any of the first mode and the second mode by operating operation portion 52.

### Fourth Embodiment

A program that causes a computer to perform processing as described with reference to the flowchart in FIG. 9 can be provided in the embodiment described above.

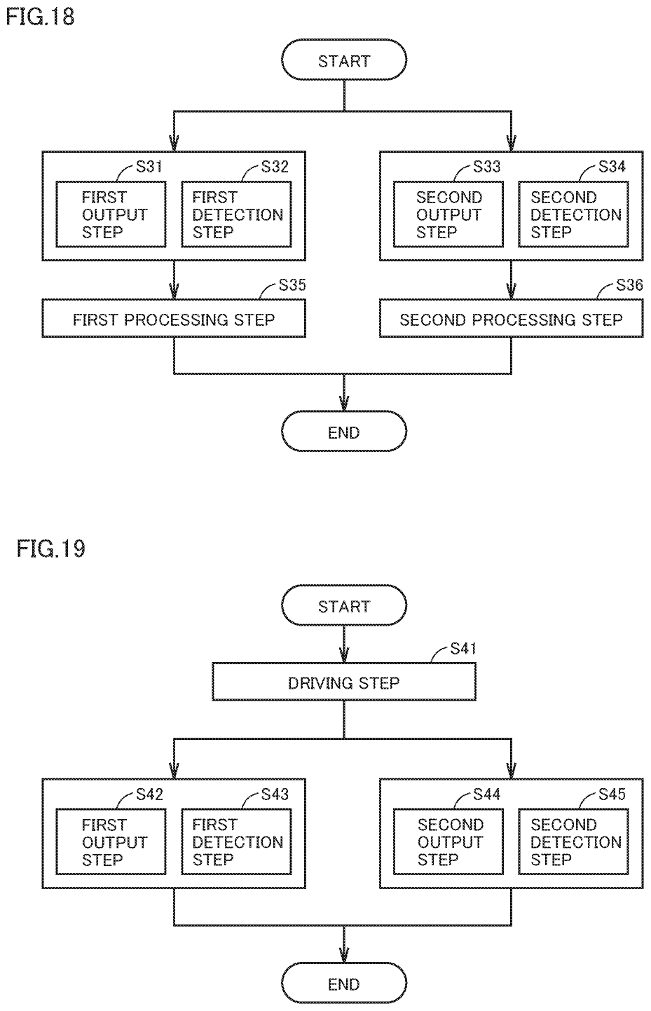

FIG. 18 is a flowchart showing a method of controlling blood pressure monitor 1 according to a fourth embodiment. FIG. 19 is a flowchart showing another method of controlling blood pressure monitor 1 according to the fourth embodiment. In step S18 in FIG. 9, in the first embodiment, processing in accordance with the flowchart in FIG. 18 is performed, and in the second embodiment, processing in accordance with the flowchart in FIG. 19 is performed.

Referring to FIG. 18, CPU 100 controls sensor units 40 and 40A in step S18 as below. Initially, the CPU performs a first output step (step S31) of controlling AC power supply unit 492 of the first pulse wave sensor unit (sensor unit 40) to output a first current signal having the first frequency to a corresponding measurement site (a measurement site corresponding to radial artery 91), a first detection step (step S32) of controlling voltage detector 492 of the first pulse wave sensor unit to detect a voltage signal representing pulse waves at the measurement site corresponding to the first pulse wave sensor unit (measurement site corresponding to radial artery 91), a second output step (step S33) of controlling AC power supply unit 492A of the second pulse wave sensor unit (sensor unit 40A) to output a second current signal having the second frequency to a corresponding measurement site (a measurement site corresponding to ulnar artery 91A), a second detection step (step S34) of controlling voltage detector 491A of the second pulse wave sensor unit to detect a voltage signal representing pulse waves at the measurement site corresponding to the second pulse wave sensor unit (measurement site corresponding to ulnar artery 91A), a first processing step (step S35) of processing the voltage signal representing pulse waves and detected in the first detection step (step S32) by using filter unit 493 based on a filter characteristic corresponding to the first frequency, and a second processing step (step S36) of processing the voltage signal representing pulse waves and detected in the second detection step by using filter unit 493A based on a filter characteristic corresponding to the second frequency.

Referring to FIG. 19, CPU 100 controls sensor units 40 and 40A in step S18 as below. The CPU initially performs a step (step S41) of controlling switching unit 150 to alternately drive the first pulse wave sensor unit (sensor unit 40) and the second pulse wave sensor unit (sensor unit 40A) at predetermined intervals, a first output step (step S42) of controlling AC power supply unit 492 of the first pulse wave sensor unit to output a first current signal having the first frequency to a corresponding measurement site (a measurement site corresponding to radial artery 91), a first detection step (step S43) of controlling voltage detector 491 of the first pulse wave sensor unit to detect a voltage signal representing pulse waves at the corresponding measurement site, a second output step (step S44) of controlling AC power supply unit 492A of the second pulse wave sensor unit to output a second current signal having the second frequency to a corresponding measurement site (a measurement site corresponding to ulnar artery 91A), and a second detection step (step S45) of controlling AC power supply unit 492A of the second pulse wave sensor unit to detect a voltage signal representing pulse waves at the corresponding measurement site.

The program can also be provided as being recorded in a non-transitory computer readable recording medium such as a compact disk read only memory (CD-ROM), a secondary storage, a main storage, and a memory card accompanying the computer of blood pressure monitor 1 in accordance with the flowcharts in FIGS. 9, 18, and 19. Alternatively, a program can also be provided as being recorded in a recording medium such as a hard disk embedded in a computer. Alternatively, a program can also be provided by downloading through network 900.

According to the present disclosure, information on pulse waves can more accurately be obtained.

It should be understood that the embodiments disclosed herein are illustrative and non-restrictive in every respect. The scope of the present invention is defined by the terms of the claims rather than the description above and is intended to include any modifications within the scope and meaning equivalent to the terms of the claims.

What is claimed is:

1. A measurement apparatus for measuring pulse waves comprising:
a first pulse wave sensor and a second pulse wave sensor that can be arranged in correspondence with respective measurement sites distant from each other; and
a hardware processor,
the first pulse wave sensor including a power-supply that outputs a current signal having a first frequency to a corresponding measurement site and a first detector that detects a voltage signal representing pulse waves from the corresponding measurement site based on a filter characteristic corresponding to the first frequency,
the second pulse wave sensor including a power-supply that outputs a current signal having a second frequency different from the first frequency to a corresponding measurement site and a second detector that detects a voltage signal representing pulse waves from the corresponding measurement site based on a filter characteristic corresponding to the second frequency,
wherein the hardware processor is configured to detect a pulse wave velocity from at least one of the pulse waves represented by the voltage signal detected by the first detector and the pulse waves represented by the voltage signal detected by the second detector, and calculate at least one of a first blood pressure based on the pulse wave velocity calculated based on the pulse waves represented by the voltage signal detected by the first detector and a second blood pressure based on the pulse wave velocity calculated based on the pulse waves represented by the voltage signal detected by the second detector.

2. The measurement apparatus according to claim 1, wherein
60 kHz is defined as the first frequency and 50 kHz is defined as the second frequency.

3. The measurement apparatus according to claim 1, wherein
50 kHz or 60 kHz is defined as the first frequency, and
50 kHz or 60 kHz is defined as the second frequency.

4. The measurement apparatus according to claim 1, further comprising a detector that detects an S/N ratio for each of the voltage signals representing the pulse waves and detected by the first detector and the second detector.

5. The measurement apparatus according to claim 4, wherein
the hardware processor is further configured to calculate a blood pressure based on the pulse wave velocity calculated based on the pulse waves represented by a voltage signal higher in S/N ratio, of the voltage signals representing the pulse waves and detected by the first detector and the second detector.

6. The measurement apparatus according to claim 5, wherein
the hardware processor is further configured to calculate a representative blood pressure, of the first blood pressure and the second blood pressure.

7. The measurement apparatus according to claim 6, wherein
the representative blood pressure includes an average blood pressure of the first blood pressure and the second blood pressure.

8. The measurement apparatus according to claim 7, wherein
the average blood pressure is represented as an average calculated with each of the first blood pressure and the second blood pressure being weighted, and
a weight for the first blood pressure is based on a corresponding S/N ratio and a weight for the second blood pressure is based on a corresponding S/N ratio.

9. The measurement apparatus according to claim 1, further comprising:
a communication circuit that communicates with an external information processing apparatus including a display, wherein
the measurement apparatus transmits a blood pressure value calculated by the hardware processor through the communication circuit to the information processing apparatus for display.

10. A measurement apparatus for measuring pulse waves comprising:
a first pulse wave sensor and a second pulse wave sensor that can be arranged in correspondence with respective measurement sites distant from each other; and
a hardware processor,
the first pulse wave sensor including a power-supply that outputs a current signal having a first frequency to a corresponding measurement site and a first detector that detects a voltage signal representing pulse waves from the corresponding measurement site,
the second pulse wave sensor including a power-supply that outputs a current signal having a second frequency to a corresponding measurement site and a second detector that detects a voltage signal representing pulse waves from the corresponding measurement site,
wherein the hardware processor is configured to:
alternately drive the first pulse wave sensor and the second pulse wave sensor at predetermined intervals,
detect a pulse wave velocity from at least one of the pulse waves represented by the voltage signal detected by the first detector and the pulse waves represented by the voltage signal detected by the second detector, and
calculate at least one of a first blood pressure based on the pulse wave velocity calculated based on the pulse waves represented by the voltage signal detected by the first detector and a second blood pressure based on the pulse wave velocity calculated based on the pulse waves represented by the voltage signal detected by the second detector.

11. The measurement apparatus according to claim 10, wherein
the first frequency and the second frequency are equal to each other.

12. The measurement apparatus according to claim 10, wherein
the first frequency is different from the second frequency.

13. The measurement apparatus according to claim 10, wherein
50 kHz or 60 kHz is defined as the first frequency, and
50 kHz or 60 kHz is defined as the second frequency.

14. The measurement apparatus according to claim 10, further comprising a detector that detects an S/N ratio for each of the voltage signals representing the pulse waves and detected by the first detector and the second detector.

15. The measurement apparatus according to claim 14, wherein
the hardware processor is further configured to calculate a blood pressure based on the pulse wave velocity calculated based on the pulse waves represented by a voltage signal higher in S/N ratio, of the voltage signals representing the pulse waves and detected by the first detector and the second detector.

16. The measurement apparatus according to claim 14, wherein
the hardware processor is further configured to calculate a representative blood pressure, of the first blood pressure and the second blood pressure.

17. The measurement apparatus according to claim 16, wherein
the representative blood pressure includes an average blood pressure of the first blood pressure and the second blood pressure.

18. The measurement apparatus according to claim 14, wherein
the average blood pressure is represented as an average calculated with each of the first blood pressure and the second blood pressure being weighted, and
a weight for the first blood pressure is based on a corresponding S/N ratio and a weight for the second blood pressure is based on a corresponding S/N ratio.

19. The measurement apparatus according to claim 10, further comprising:
a communication circuit that communicates with an external information processing apparatus including a display unit, wherein
the measurement apparatus transmits a blood pressure value calculated by the hardware processor through the communication circuit to the information processing apparatus for display.

20. A non-transitory computer-readable recording medium having a program stored thereon, the program having a computer perform a method of measuring by using an apparatus comprising a first pulse wave sensor and a second pulse wave sensor that can be arranged in correspondence with respective measurement sites distant from each other,
the method including:
controlling the first pulse wave sensor to output a current signal having a first frequency to a corresponding measurement site,
controlling the first pulse wave sensor to detect a voltage signal representing pulse waves from the corresponding measurement site based on a filter characteristic corresponding to the first frequency,
controlling the second pulse wave sensor to output a current signal having a second frequency different from the first frequency to a corresponding measurement site,
controlling the second pulse wave sensor to detect a voltage signal representing pulse waves from the corresponding measurement site based on a filter characteristic corresponding to the second frequency,
detecting a pulse wave velocity from at least one of the pulse waves represented by the voltage signal detected by the first pulse wave sensor and the pulse waves represented by the voltage signal detected by the second pulse wave sensor, and
calculating at least one of a first blood pressure based on the pulse wave velocity calculated based on the pulse waves represented by the voltage signal detected by the first pulse wave sensor and a second blood pressure based on the pulse wave velocity calculated based on the pulse waves represented by the voltage signal detected by the second pulse wave sensor.

21. A non-transitory computer-readable recording medium having a program stored thereon, the program having a computer perform a method of measuring by using an apparatus comprising a first pulse wave sensor and a second pulse wave sensor that can be arranged in correspondence with respective measurement sites distant from each other,
the method including:
controlling the first pulse wave sensor to output a current signal having a first frequency to a corresponding measurement site,
controlling the first pulse wave sensor to detect a voltage signal representing pulse waves from the corresponding measurement site,
controlling the second pulse wave sensor to output a current signal having a second frequency to a corresponding measurement site,
controlling the second pulse wave sensor to detect a voltage signal representing pulse waves from the corresponding measurement site,
alternately driving the first pulse wave sensor and the second pulse wave sensor at predetermined intervals,
detecting a pulse wave velocity from at least one of the pulse waves represented by the voltage signal detected by the first pulse wave sensor and the pulse waves represented by the voltage signal detected by the second pulse wave sensor, and
calculating at least one of a first blood pressure based on the pulse wave velocity calculated based on the pulse waves represented by the voltage signal detected by the first pulse wave sensor and a second blood pressure based on the pulse wave velocity calculated based on the pulse waves represented by the voltage signal detected by the second pulse wave sensor.
